(19) United States
(12) Patent Application Publication
(10) Pub. No.: US 2025/0022566 A1
(43) Pub. Date: Jan. 16, 2025

(54) METHOD FOR CLASSIFYING A MEDICAL DEVICE AND/OR DRUG, SYSTEM AND TRAINING METHOD

(71) Applicant: BIOTRONIK AG, Buelach (CH)

(72) Inventor: Azadeh Mehrabi, Meilen (CH)

(21) Appl. No.: 18/714,365

(22) PCT Filed: Nov. 29, 2022
(86) PCT No.: PCT/EP2022/083569

§ 371 Date: May 29, 2024

(30) Foreign Application Priority Data
Dec. 22, 2021 (EP) 21216804.1

(51) Int. Cl. G16H 20/10; G16H 10/60; G16H 20/40; G16H 50/70

(52) U.S. Cl. CPC G16H 20/10; G16H 10/60; G16H 20/40; G16H 50/70

(57) ABSTRACT

A computer implemented method for classifying or suggesting at least one medical device and/or at least one drug clinically associated with a first data set (DS1) of medical parameters of a patient, The method includes providing (S1) the first data set (DS1), applying (S2) a machine learning algorithm (A1) and/or a rule-based algorithm (A2) to the first data set (DS1), and outputting (S3) a second data set (DS2) including at least one class (C) representing the at least one medical device and/or the at least one drug clinically associated with the first data set (DS1) The invention further relates to a corresponding system and training method.

### PRIORITY CLAIM

This application is a 35 U.S.C. 371 US National Phase and claims priority under 35 U.S.C. § 119, 35 U.S.C. 365 (b) and all applicable statutes and treaties from prior PCT Application PCT/EP2022/083569, which was filed Nov. 29, 2022, which application claimed priority from EP application Ser. No. 21/216,804.1, which was filed Dec. 22, 2021.

### FIELD OF THE INVENTION

The invention relates to a computer implemented method for classifying or suggesting at least one medical device, at least one treatment, and/or at least one drug clinically associated with a first data set of medical parameters of a patient.

### BACKGROUND

Furthermore, the invention relates to a computer-implemented method for providing a trained machine learning algorithm for classifying or suggesting at least one medical device, at least one treatment, and/or at least one drug clinically associated with a first data set of medical parameters of a patient.

In addition, the invention relates to a system for classifying or suggesting at least one medical device, at least one treatment, and/or at least one drug clinically associated with a first data set of medical parameters of a patient.

Medical practitioners when diagnosing patient conditions customarily rely on professional experience and/or existing medical records of the patient to be diagnosed. Since a health condition of the patient however depends on a plurality of factors such as age, gender, diet, lifestyle, physical activity, patient symptoms as well as medical parameters and comorbidities, successfully diagnosing a specific health condition requires to take all of the aforementioned factors into consideration.

Furthermore, there are a plurality of medical devices such as implantable medical devices as well as treatments or drugs that may be used for treating a specific medical condition diagnosed by the medical practitioner. Which of said plurality of medical devices, treatments and/or drugs is best suited to the individual patient is not always straightforward to determine.

It is an idea of the present invention to use information from e.g. clinical trials to enable enhanced data-driven decision making for medical practitioners when diagnosing medical conditions of patients and deciding the usage of suitable medical devices, treatments, and/or drugs to treat the medical condition. Possible data sources for said medical data is clinical trial data and data of previous diagnoses of medical practitioners.

### SUMMARY OF THE INVENTION

A preferred computer implemented method for classifying or suggesting at least one medical device and/or at least one drug clinically associated with a first data set (DS1) of medical parameters of a patient includes providing (S1) the first data set (DS1), applying (S2) a machine learning algorithm (A1) and/or a rule-based algorithm (A2) to the first data set (DS1), and outputting (S3) a second data set (DS2) including at least one class (C) representing the at least one medical device and/or the at least one drug clinically associated with the first data set (DS1) The invention further relates to a corresponding system and training method. Methods and systems of the invention can aid to determine a specific medical device, treatment and/or drug best suited for a specific patient condition.

### BRIEF DESCRIPTION OF THE DRAWINGS

For a more complete understanding of the present invention and advantages thereof, reference is now made to the following description taken in conjunction with the accompanying drawings. The invention is explained in more detail below using exemplary embodiments, which are specified in the schematic figures of the drawings, in which:

FIG. 1 shows a flowchart of a computer implemented method for classifying or suggesting at least one medical device, at least one treatment, and/or at least one drug clinically associated with a first data set of medical parameters of a patient according to a preferred embodiment of the invention;

FIG. 2 shows a flowchart of a computer implemented method for providing a trained machine learning algorithm for classifying or suggesting at least one medical device, at least one treatment, and/or at least one drug clinically associated with a first data set of medical parameters of a patient according to the preferred embodiment of the invention; and

FIG. 3 shows a schematic illustration of a system for classifying at least one medical device and/or at least one drug clinically associated with a first data set of medical parameters of a patient according to the preferred embodiment of the invention.

### DETAILED DESCRIPTION OF THE PREFERRED EMBODIMENTS

The present invention provides a computer implemented method for classifying or suggesting at least one medical device, at least one treatment, and/or at least one drug clinically associated with a first data set of medical parameters of a patient.

The method includes providing a first data set of medical parameters of a patient and providing a plurality of third data sets of medical parameters of further patients and applying a machine learning algorithm and/or a rule-based algorithm to the first data set of medical parameters of the patient and to the plurality of third data sets of medical parameters of further patients for classifying or suggesting a medical device, in particular an implantable medical device, and/or a drug clinically associated with the first data set of medical parameters of the patient.

Furthermore, the method includes outputting a second data set including at least one class representing and particularly the at least one medical device, the at least one treatment, and/or the at least one drug clinically associated with the first data set of medical parameters of the patient.

The present invention further provides a computer-implemented method for providing a trained machine learning algorithm for classifying or suggesting at least one medical device, at least one treatment, and/or at least one drug clinically associated with a first data set of medical parameters of a patient.

The method includes providing a first training data set including a first data set of medical parameters of a patient and providing a second training data set including a second data set including at least one class representing the at least one medical device, the at least one treatment, and/or the at least one drug clinically associated with the first data set of medical parameters of the patient.

In addition, the method includes training the machine learning algorithm by an optimization algorithm which calculates an extreme value or threshold value of a loss function for classifying or suggesting at least one medical device, the at least one treatment, and/or at least one drug clinically associated with a first data set of medical parameters of a patient.

The present invention further provides a system for classifying or suggesting at least one medical device, at least one treatment, and/or at least one drug clinically associated with a first data set of medical parameters of a patient.

The system includes means for providing a first data set of medical parameters of a patient, means for providing a plurality of third data sets of medical parameters of further patients and means for applying a machine learning algorithm and/or a rule-based algorithm to the first data set of medical parameters of the patient for classifying or suggesting a medical device, in particular an implantable medical device, a treatment, and/or drug clinically associated with the first data set of medical parameters of the patient.

The means for providing a first data set of medical parameters of a patient may be a mobile device including an application or a first computer, and the means for providing a plurality of third data sets of medical parameters of further patients may be a server or a second computer including a database.

Alternatively, the system may include means for providing the first data set of medical parameters of a patient and the plurality of third data sets of medical parameters of further patients.

Moreover, the system includes means for outputting a second data set including at least one class representing the at least one medical device, the at least one treatment, and/or the at least one drug clinically associated with the first data set of medical parameters of the patient.

The outputted second data set can e.g. be presented to the medical practitioner on an app installed on a mobile device. The app or the mobile device may also be used to provide a first data set of medical parameters of a patient, e.g. by the patient or the medical practitioner, as well as to provide general information and medical history.

The at least one medical device, the at least one treatment, and/or the at least one drug being clinically associated with the first data set of medical parameters of a patient refers to the fact that the machine learning algorithm and/or the rule-based algorithm determines, based on medical data of other patients, what medical device, individual treatment and/or drug is best suited for the patient, i.e. is the input data of the respective algorithm clinically associated to the output data, i.e. the class representing the medical device, treatment, and/or drug.

Machine learning algorithms are based on using statistical techniques to train a data processing system to perform a specific task without being explicitly programmed to do so. The goal of machine learning is to construct algorithms that can learn from data and make predictions. These algorithms create mathematical models that can be used, for example, to classify data or to solve regression type problems.

According to an aspect of the invention, the first data set of medical parameters of the patient include text-based medical data and image-based medical data, and the plurality of third data sets of medical parameters of further patients includes text-based medical data, image-based medical data and medical treatment data, wherein the text-based medical data include an age, a gender, a weight, comorbidities, a drug prescription history, a treatment history, a heart rate, a blood pressure, an activity profile and/or symptoms of the patient, and wherein the image-based medical data includes a CT-scan, a MRI-scan, an angiograph, and/or at least one ultrasound-image, and wherein the medical treatment data includes data identifying a medical device, in particular an implantable medical device, a treatment, and/or a drug.

The medical treatment data includes data identifying the medical device, in particular an implantable medical device, the treatment, and/or the drug which has/have been used for medical treatment of the further patient associated with the third data set of medical parameters. In some examples, the medical treatment data may further include a treatment success rate, a medical device success rate and/or a drug success rate. The medical treatment data may include all of the data available by clinical trials, e.g. a medical treatment history of the patient.

Alternatively or additionally, the text-based medical data of the plurality of third data sets of medical parameters of further patients may include the medical treatment data.

Using said medical parameters, the machine learning algorithm and/or a rule-based algorithm can thus determine a suitable medical device, treatment, and/or drug based on the plurality of third data sets of medical parameters of further patients, e.g. clinical trial data, wherein the respective algorithm either identifies nonlinear relations or compares the data set of the patient to the plurality of third data sets of medical parameters of further patients, e.g. the assets of other patients included by the clinical trial data. Particularly, machine learning algorithm and/or a rule-based algorithm classifies or groups the medical parameters of the plurality of third data sets of medical parameters of further patients, e.g. from clinical trials, of cohorts of patients having a similar medical condition, which can thus advantageously serve as a reference for aiding the decision of the medical practitioner on which treatment, drug and/or medical device to choose for the present patient.

According to a further aspect of the invention, the rule-based algorithm compares the provided first data set of medical parameters of the patient with the plurality of third data sets of medical parameters of further patients, wherein each of the third data sets of medical parameters of further patients is related to or includes at least one class representing at least one medical device, the at least one treatment, and/or at least one drug clinically associated with the third data set of medical parameters of the further patients. The rule-based algorithm thus advantageously identifies suitable medical devices, treatments, and/or drugs based on the comparison result.

In some examples, the machine learning algorithm, applied to the plurality of third data sets of medical parameters of further patients, may determine the at least one class representing at least one medical device, the at least one treatment, and/or at least one drug clinically associated with each third data set of medical parameters of the further patients.

According to a further aspect of the invention, the machine learning algorithm and/or a rule-based algorithm outputs the at least one class representing the at least one medical device, the at least one treatment, and/or the at least one drug clinically associated with the first data set of medical parameters of the patient having a closest match to the plurality of third data sets of medical parameters of further patients. The medical practitioner thus advantageously receives a recommendation for using a medical device, administering a treatment and/or prescribing a particular drug providing a best match to the medical condition of the present patient.

According to a further aspect of the invention, the at least one class representing the at least one medical device, the at least one treatment, and/or the at least one drug clinically associated with the first data set of medical parameters of the patient is outputted by the machine learning algorithm and/or a rule-based algorithm in an order of similarity to the plurality of third data sets of medical parameters of further patients. The medical practitioner thus receives a plurality of results structured by order of similarity hence facilitating decision-making.

According to a further aspect of the invention, the machine learning algorithm and/or the rule-based algorithm includes a first algorithm applied to the text-based medical data of the first data set of medical parameters of the patient and of the plurality of third data sets of medical parameters of further patients and a second algorithm applied to the image-based medical data of the first data set of medical parameters of the patient and of the plurality of third data sets of medical parameters of further patients, wherein the first algorithm outputs at least a first numeric value to which a first score is assigned, and wherein and the second algorithm outputs at least a second numeric value to which a second score is assigned. The text-based medical data and the image-based medical data can thus be assigned a specific weight or score in order to enhance accuracy of the overall classification or suggestion result.

According to a further aspect of the invention, the second data set including the at least one class representing the at least one medical device, the at least one treatment, and/or the at least one drug clinically associated with the first data set of medical parameters of the patient may be calculated by forming a weighted average from a sum product including a first product of the first numeric value and the assigned first score, and a second product of the second numeric value and the assigned second score. The text-based information and the image-based information can thus advantageously be combined into an overall score.

According to a further aspect of the invention, the machine learning algorithm and/or the rule-based algorithm outputs a third numeric value representing a number of patients using the medical device, in particular the implantable medical device, the at least one treatment, and/or the drug clinically associated with the first data set of medical parameters of the patient and text-based information indicating a patient outcome using the medical device and/or the drug for a predetermined amount of time. A number of other patients using the medical device, the treatment and/or taking the drug can thus be presented to the medical practitioner as additional data points on which a decision can be based.

The above-mentioned number of patients may be advantageously grouped or selected by a specified medical condition or treatment background, such as for example diabetes, heart failure, obesity, and the like. For example, one third numeric value may represent patients having a diabetes indication, a further third numeric value may represent patients having a heart failure indication or history and so on. In addition, the medical parameters of the patients having a similar medical condition can further serve as further data points aiding the decision of the medical practitioner on which medical device, treatment or drug to choose for the present patient.

Additionally or alternatively, the machine learning algorithm and/or the rule-base algorithm may present at least one further patient having a close matching, particularly the closest matching, to the first data set of the patient to the medical practitioner as additional data points on which a decision can be based, wherein the at least one further patient having the close or closest matching has been identified or classified by the machine learning algorithm and/or the rule-base algorithm.

According to a further aspect of the invention, the machine learning algorithm and/or the rule-based algorithm outputs a fourth numeric value representing a probability of a successful patient outcome using the medical device, in particular the implantable medical device, the treatment, and/or the drug clinically associated with the first data set of medical parameters of the patient. The medical practitioner can with this information be additionally supported to evaluate a probability of success of using the medical device, the treatment and/or prescribing a drug for this particular patient.

According to a further aspect of the invention, the machine learning algorithm and/or the rule-based algorithm outputs fifth numeric value representing if the at least one medical device, the at least one treatment, and/or the at least one drug clinically associated with the first data set of medical parameters of the patient is suitable for a current stage of a health condition of the patient. Not only a particular health condition but also the current stage of said calculation of the patient can thus be taken into account and compared to data points of other patients.

According to a further aspect of the invention, in response to outputting the second data set including at least one class representing the at least one medical device, the at least one treatment, and/or the at least one drug clinically associated with the first data set of medical parameters of the patient, a medical practitioner information request is triggered requesting if the medical practitioner agrees or disagrees with using the at least one medical device, the at least one treatment, and/or the at least one drug clinically associated with the first data set of medical parameters of the patient for patient treatment.

The feedback of the medical practitioner, i.e. based on the professional experience of the medical practitioner, can thus advantageously enhance a decision making of the medical practitioner while at the same time improving the quality of the medical data for future classifications.

According to a further aspect of the invention, if in response to the medical practitioner information request disagreement with using the at least one medical device, the at least one treatment, and/or the at least one drug clinically associated with the first data set of medical parameters of the patient for patient treatment is expressed, a further medical practitioner information request is triggered requesting to provide reasons for the disagreement. This feedback thus serves to further train the algorithm, e.g. a medical practitioner can state that a particular drug, medical device, or treatment is not suitable for an age group of a particular patient or with a specific medical history.

According to a further aspect of the invention, a response to the medical practitioner information request is used to train the machine learning algorithm and/or update rules of the rule-based algorithm. The machine learning algorithm or the rule-based algorithm can thus be trained to enhance their performance for future classifications.

The herein described features of the computer implemented method for classifying or suggesting at least one medical device, at least one treatment, and/or at least one drug clinically associated with a first data set of medical parameters of a patient are also disclosed for the system for classifying or suggesting at least one medical device, at least one treatment, and/or at least one drug clinically associated with a first data set of medical parameters of a patient and vice versa.

The computer implemented method of FIG. 1 for classifying at least one medical device and/or at least one drug clinically associated with a first data set DS1 of medical parameters of a patient includes providing S1 a first data set DS1 of medical parameters of a patient and providing a plurality of third data sets DS3 of medical parameters of further patients.

Furthermore, the method includes applying S2 a machine learning algorithm A1 and/or a rule-based algorithm A2 to the first data set DS1 of medical parameters of the patient and to the plurality of third data sets DS3 of medical parameters of further patients for classifying or suggesting a medical device, in particular an implantable medical device, a treatment and/or drug clinically associated with the first data set DS1 of medical parameters of the patient. The machine learning algorithm A1 and/or the rule-based algorithm A2 may be subsequently and separately applied to the first data set DS1 of medical parameters of the patient and/or to the plurality of third data sets DS3 of medical parameters of further patients.

In addition, the method includes outputting S3 a second data set DS2 including at least one class C representing the at least one medical device and/or the at least one drug clinically associated with the first data set DS1 of medical parameters of the patient.

The first data set DS1 of medical parameters of the patient include text-based medical data DS1a and image-based medical data DS1b. The text-based medical data DS1a include an age, a gender, a weight, comorbidities, a drug prescription history, treatment history, a heart rate, a blood pressure, an activity profile and/or symptoms of the patient. Furthermore, the image-based medical data DS1b includes a CT-scan, a MRI-scan and/or at least one ultrasound-image.

Each of the plurality of third data sets DS3 of medical parameters of further patients includes text-based medical data DS3a, image-based medical data DS3b and medical treatment data DS3c. The text-based medical data DS3a include an age, a gender, a weight, comorbidities, a drug prescription history, treatment history, a heart rate, a blood pressure, an activity profile and/or symptoms of the patient. Furthermore, the image-based medical data DS3b includes a CT-scan, a MRI-scan and/or at least one ultrasound-image. The medical treatment data DS3c includes data identifying a medical device, a treatment, and/or a drug which has/have been used for medical treatment of the further patient associated with the third data set of medical parameters.

The machine learning algorithm A1 and/or rule-based algorithm A2 compares the provided first data set DS1 of medical parameters of the patient with a plurality of third data sets DS3 of medical parameters of further patients. Each of the third data sets DS3 of medical parameters of further patients is related to or includes at least one class C representing at least one medical device at least one treatment, and/or at least one drug clinically associated with the third data set DS3 of medical parameters of the further patients.

The machine learning algorithm A1 and/or a rule-based algorithm A2 outputs the at least one class C representing the at least one medical device, the at least one treatment, and/or the at least one drug clinically associated with the first data set DS1 of medical parameters of the patient having a closest match to the plurality of third data sets DS3 of medical parameters of further patients.

The at least one class C representing the at least one medical device and/or the at least one drug clinically associated with the first data set DS1 of medical parameters of the patient is outputted by the machine learning algorithm A1 and/or the rule-based algorithm A2 in an order of similarity to the plurality of third data sets DS3 of medical parameters of further patients.

The machine learning algorithm A1 and/or the rule-based algorithm A2 includes a first algorithm applied to the text-based medical data DS1a and DS3a and a second algorithm applied to the image-based medical data DS1b and DS3b. Moreover, the first algorithm outputs at least a first numeric value 10 to which a first score 12 is assigned. In addition, the second algorithm outputs at least a second numeric value 14 to which a second score 16 is assigned.

The second data set DS2 including the at least one class C representing the at least one medical device, the at least one treatment, and/or the at least one drug clinically associated with the first data set DS1 of medical parameters of the patient is calculated by forming a weighted average from a sum product including a first product of the first numeric value 10 and the assigned first score 12, and a second product of the second numeric value 14 and the assigned second score 16.

The machine learning algorithm A1 and/or the rule-based algorithm A2 outputs a third numeric value 18 representing a number of patients using the medical device, in particular the implantable medical device, the treatment, and/or the drug clinically associated with the first data set DS1 of medical parameters of the patient and text-based information 20 indicating a patient outcome using the medical device, the treatment, and/or the drug for a predetermined amount of time.

Furthermore, the machine learning algorithm A1 and/or the rule-based algorithm A2 outputs a fourth numeric value 22 representing a probability of a successful patient outcome using the medical device, in particular the implantable medical device, the treatment, and/or the drug clinically associated with the first data set DS1 of medical parameters of the patient.

Moreover, the machine learning algorithm A1 and/or the rule-based algorithm A2 outputs a fifth numeric value 24 representing if the at least one medical device, the at least one treatment, and/or the at least one drug clinically associated with the first data set DS1 of medical parameters of the patient is suitable for a current stage of a health condition of the patient.

In response to outputting the second data set DS2 including at least one class C representing the at least one medical device, the at least one treatment, and/or the at least one drug clinically associated with the first data set DS1 of medical parameters of the patient a medical practitioner information request R1 is triggered requesting if the medical practitioner agrees or disagrees with using the at least one medical device, the at least one treatment, and/or the at least one drug clinically associated with the first data set DS1 of medical parameters of the patient for patient treatment.

If in response to the medical practitioner information request R1 disagreement with using the at least one medical device, the at least one treatment, and/or the at least one drug clinically associated with the first data set DS1 of medical parameters of the patient for patient treatment is expressed, a further medical practitioner information request R2 is triggered requesting to provide reasons for the disagreement.

Furthermore, a response to the medical practitioner information request R1 is used to train the machine learning algorithm A1 and/or update rules of the rule-based algorithm A2.

The medical practitioner information request R1 and a further medical practitioner information request R2 are sent to and displayed by an app 26 on a mobile device or via a web-based app.

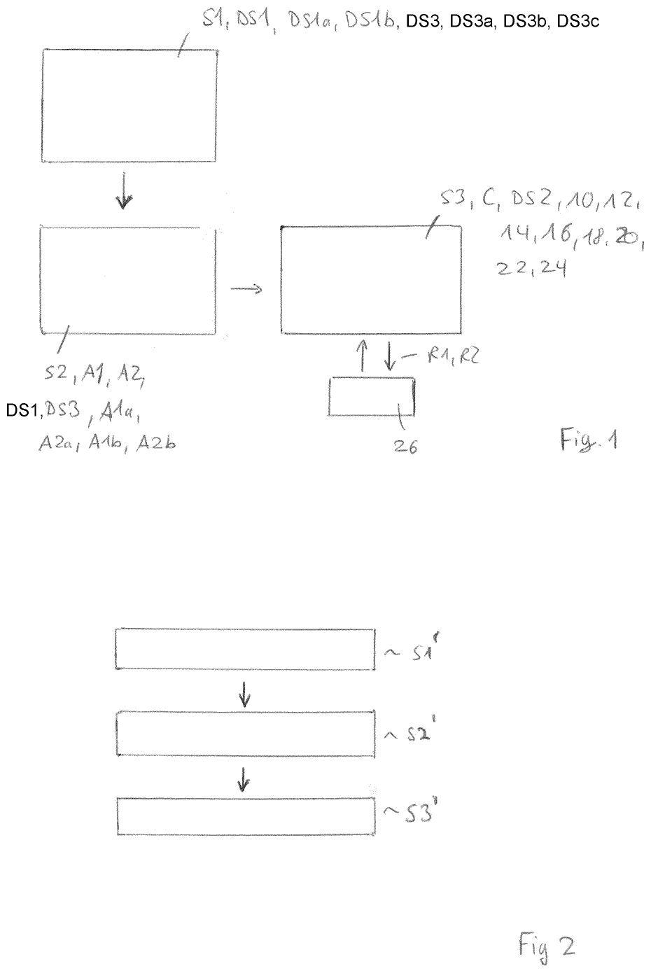

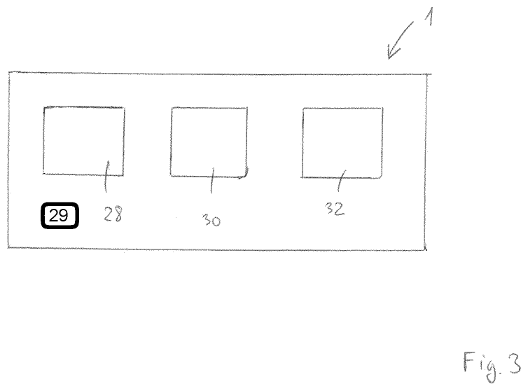

FIG. 2 shows a flowchart of a computer implemented method for providing a trained machine learning algorithm A1 for classifying or suggesting at least one medical device, the at least one treatment, and/or at least one drug clinically associated with a first data set DS1 of medical parameters of a patient according to the preferred embodiment of the invention.

The method includes providing S1′ a first training data set including a first data set DS1 of medical parameters of a patient and providing S2′ a second training data set including a second data set DS2 including at least one class C representing the at least one medical device and/or the at least one drug clinically associated with the first data set DS1 of medical parameters of the patient.

Furthermore, the method includes training S3′ the machine learning algorithm A1 by an optimization algorithm which calculates an extreme value or threshold value of a loss function for classifying or suggesting at least one medical device, the at least one treatment, and/or at least one drug clinically associated with a first data set DS1 of medical parameters of a patient.

FIG. 3 shows a schematic illustration of a system for classifying or suggesting at least one medical device, at least one treatment, and/or at least one drug clinically associated with a first data set DS1 of medical parameters of a patient according to the preferred embodiment of the invention.

The system includes code and hardware 28 for providing a first data set DS1 of medical parameters of a patient and for providing a plurality of third data sets DS3 of medical parameters of further patients as well as code and hardware 30 for applying a machine learning algorithm A1 and/or a rule-based algorithm A2 to the first data set DS1 of medical parameters of the patient and to the plurality of third data sets DS3 of medical parameters of further patients for classifying or suggesting a medical device, in particular an implantable medical device, a treatment and/or a drug clinically associated with the first data set DS1 of medical parameters of the patient.

In addition, the system includes code and hardware 32 for outputting a second data set DS2 including at least one class C representing the at least one medical device, the at least one treatment, and/or the at least one drug clinically associated with the first data set DS1 of medical parameters of the patient.

In addition, the system can further output comments made by medical practitioners as to why a particular drug, treatment or medical device is suitable or not suitable for a specific condition. The system can thus provide a personalized treatment plan for a particular patient based on accurate data of all available data points in the database.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

### LIST OF REFERENCE NUMERALS

1 system
10 first numeric value
12 first score
14 second numeric value
16 second score
18 third numeric value
20 text-based information
22 fourth numeric value
24 fifth numeric value
26 app
28 code and hardware
30 code and hardware
32 code and hardware
A1 machine learning algorithm
A1a first algorithm
A1b second algorithm
A2 rule-based algorithm
A2a first algorithm
A2b second algorithm
C class
DS1 first data set
Ds1a text-based medical data
DS2 second data set
DS1b image-based medical data
DS3 third data set
R1 medical practitioner information request
R2 further medical practitioner information request
S1-S3 method steps
S1′-S3′ method steps

1. A computer implemented method for classifying or suggesting at least one medical device, at least one treatment, and/or at least one drug clinically associated with a first data set (DS1) of medical parameters of a patient, comprising the steps of:
providing (S1) the first data set (DS1);
providing a plurality of third data sets (DS3) of medical parameters of other patients;
applying (S2) a machine learning algorithm (A1) and/or a rule-based algorithm (A2) to the first data set (DS1) and to the plurality of third data sets (DS3) to classify or suggest a medical device, in particular an implantable medical a treatment and/or drug clinically associated with the first data set (DS1) of medical parameters of the patient; and
outputting (S3) a second data set (DS2) comprising at least one class (C) representing the at least one medical device, the at least one treatment, and/or the at least one drug clinically associated with the first data set (DS1).

2. The computer implemented method of claim 1, wherein the first data set (DS1) comprises text-based medical data (DS1a) and image-based medical data (DS1b), wherein the plurality of third data sets (DS3) comprises text-based medical data (DS3a) and image-based medical data (DS3b), medical treatment data (DS3c), wherein the text-based medical data (DS1a, DS3a) comprise an age, a gender, a weight, comorbidities, a drug prescription history, a treatment history, a heart rate, a blood pressure, an activity profile and/or symptoms of the patient, and wherein the image-based medical data (DS1b, DS3b) comprises a CT-scan, a MRI-scan, an angiograph and/or at least one ultrasound-image, and wherein the medical treatment data (DS3c) comprises data identifying a medical device, a treatment, and/or a drug.

3. The computer implemented method of claim 1, wherein the rule-based algorithm (A2) compares the first data set (DS1) with the plurality of third data sets (DS3), wherein each of the third data sets (DS3) is related to or comprises at least one class (C) representing the at least one medical device, the at least one treatment, and/or at least one drug.

4. The computer implemented method of claim 3, wherein the machine learning algorithm (A1) and/or the rule-based algorithm (A2) outputs the at least one class (C) with the first data set (DS1) having a closest match to the plurality of third data sets (DS3).

5. The computer implemented method of claim 4, wherein the at least one class (C) is outputted by the machine learning algorithm (A1) and/or the rule-based algorithm (A2) in an order of similarity to the plurality of third data sets (DS3).

6. The computer implemented method of claim 2, wherein the machine learning algorithm (A1) and/or the rule-based algorithm (A2) comprises a first algorithm (A1a; A2a) applied to the text-based medical data (DS1a, DS3a) and a second algorithm (A1b; A2b) applied to the image-based medical data (DS1b, DS3b), wherein the first algorithm (Ala; A2a) outputs at least a first numeric value to which a first score is assigned, and wherein and the second algorithm (A1b; A2b) outputs at least a second numeric value to which a second score (16) is assigned.

7. The computer implemented method of claim 6, wherein the second data set (DS2) is calculated by forming a weighted average from a sum product comprising a first product of the first numeric value and the first score, and a second product of the second numeric value and the second score.

8. The implemented method of claim 6, wherein the machine learning algorithm (A1) and/or the rule-based algorithm (A2) outputs a third numeric value representing a number of patients using the medical device, the treatment, and/or the drug clinically associated with the first data set (DS1) and text-based information indicating a patient outcome using the medical device, the treatment, and/or the drug for a predetermined amount of time.

9. The computer implemented method of claim 8, wherein the machine learning algorithm (A1) and/or the rule-based algorithm (A2) outputs a fourth numeric value representing a probability of a successful patient outcome using the medical device, the treatment, and/or the drug clinically associated with the first data set (DS1).

10. The implemented method of any one claim 9, wherein the machine learning algorithm (A1) and/or the rule-based algorithm (A2) outputs a fifth numeric value representing if the at least one medical device, the at least one treatment, and/or the at least one drug is suitable for a current stage of a health condition of the patient.

11. The computer implemented method claim 1, wherein, in response to outputting the second data set (DS2), a medical practitioner information request (R1) is triggered requesting if the medical practitioner agrees or disagrees with using the at least one medical device, the at least one treatment, and/or the at least one drug clinically associated with the first data set (DS1), of medical parameters-of-the-patient for patient treatment.

12. The computer implemented method of claim 11, wherein if in response to the medical practitioner information request (R1) disagreement with using the at least one medical device, the at least one treatment, and/or the at least one drug clinically associated with the first data set (DS1) of medical parameters of the patient for patient treatment is expressed, a further medical practitioner information request (R2) is triggered requesting to provide reasons for the disagreement.

13. The computer implemented method of claim 11, wherein a response to the medical practitioner information request (R1) is used to train the machine learning algorithm (A1) and/or update rules of the rule-based algorithm (A2).

14. A computer-implemented method for providing a trained machine learning algorithm (A1) for classifying or suggesting at least one medical device, at least one treatment, and/or at least one drug clinically associated with a first data set (DS1) of medical parameters of a patient, comprising the steps of:
providing (S1′) a first training data set comprising a first data set (DS1) of medical parameters of a patient;
providing (S2′) a second training data set comprising a second data set (DS2) comprising at least one class (C) representing the at least one medical device, the at least one treatment, and/or the at least one drug clinically associated with the first data set (DS1); and
training (S3′) the machine learning algorithm (A1) by an optimization algorithm which calculates a threshold value of a loss function for classifying or suggesting at least one medical device, the at least one treatment, and/or at least one drug clinically associated with the first data set (DS1).

15. A system for classifying or suggesting at least one medical device, at least one treatment, and/or at least one drug clinically associated with a first data set (DS1) of medical parameters of a patient, comprising:
means for providing a first data set (DS1) of medical parameters of a patient;
means for providing a plurality of third data sets (DS3) of medical parameters of other patients;
means for applying a machine learning algorithm (A1) and/or a rule-based algorithm (A2) to the first data set (DS1); and
means for outputting a second data set (DS2) comprising at least one class (C) representing the at least one medical device, the at least one treatment, and/or the at least one drug clinically associated with the first data set (DS1).
